(19) United States
(12) Patent Application Publication
(10) Pub. No.: US 2008/0220076 A1
(43) Pub. Date: Sep. 11, 2008

(54) Active Agent Formulations, Methods of Making, and Methods of Use

(75) Inventors: Kristin Anne Arnold, Morrisville, PA (US); Hengsheng Feng, Cherry Hill, NJ (US); Kurt R. Nielsen, Chadds Ford, PA (US)

Correspondence Address: CANTOR COLBURN, LLP, 20 Church Street, 22nd Floor, Hartford, CT, 06103, US

(73) Assignee: MUTUAL PHARMACEUTICAL COMPANY, INC., Philadelphia, PA (US)

(21) Appl. No.: 12/122,402

(22) Filed: May 16, 2008

Related U.S. Application Data
(63) Continuation of application No. 11/768,476, filed on Jun. 26, 2007.
(60) Provisional application No. 60/805,823, filed on Jun. 26, 2006.

(51) Int. Cl. A61K 9/14; A61K 31/454; A61K 31/4355; A61K 31/436

(52) U.S. Cl. 424/489; 514/323; 514/291

(57) ABSTRACT

Active agent compositions comprising active agent particles having an effective average particle size of less than 2000 nm, wherein the compositions comprise a particle sequestrant are disclosed. Compositions having an effective average particle size of less than 2000 nm, wherein the compositions comprise no added surfactants, phospholipids, or combinations thereof, are also disclosed.

### CROSS REFERENCE TO RELATED APPLICATION

This application is a continuation of U.S. Application Ser No. 11/768,476 filed Jun. 26, 2007, which claims priority from U.S. Provisional Application Ser. No. 60/805,823 filed Jun. 26, 2006, which is hereby incorporated by reference in its entirety.

### BACKGROUND

Bioavailability means the extent and/or rate at which an active agent is absorbed into a living system, or is made available at the site of physiological activity. Many factors can affect bioavailability including the dosage form and various properties of the active agent and/or dosage form, e.g., dissolution rate of the active agent. Poor bioavailability is a significant problem encountered in the development of pharmaceutical compositions, particularly those containing an active agent that is poorly soluble in water. Poorly water-soluble active agents can be eliminated from the gastrointestinal tract before being absorbed into the circulation. It is known that the rate of dissolution of a particulate active agent can increase with increasing surface area, i.e., decreasing particle size.

Fenofibrate is an example of an active pharmaceutical agent with poor water solubility. Fenofibrate, 2-[4-(4-chlorobenzoyl) phenoxy]-2-methyl-propanoic acid, 1-methylethyl ester, is used in the treatment of endogenous hyperlipidaemias, hypercholesterolaemias, and hypertriglyceridaemias in adults. The preparation of fenofibrate is disclosed in U.S. Pat. No. 4,058,552. Fenofibric acid, the active metabolite of fenofibrate, produces reductions in total cholesterol, LDL cholesterol, apolipoprotein B, total triglycerides and triglyceride rich lipoprotein (VLDL) in treated patients. Also, treatment with fenofibrate results in increases in high-density lipoprotein (HDL) and apoproteins apoAI and apoAII. Prolonged treatment with fenofibrate at the rate of about 300 to about 400 mg per day makes it possible to obtain a reduction in total cholesterol of about 20 to about 25%, and a reduction in the levels of triglycerides of about 40 to about 50%.

The poor water solubility of fenofibrate can limit its absorption in the gastrointestinal (GI) tract. To remedy this problem, research groups have tried a multitude of strategies including, for example, micronized fenofibrate formulations, the combination of fenofibrate and vitamin E, the use of diethylene glycol monoethyl ether (DGME) as solubilizer, and the combination of fenofibrate with one or more polyglycolyzed glycerides. Another approach has been to employ nanoparticulate fenofibrate. The pharmacokinetics parameters for nanoparticulate fenofibrate formulations, commercially available from Abbott as TriCor® 145 mg and 48 mg, are reportedly not significantly affected by the fed or fasting state of the subject.

The present invention addresses the need for improved fenofibrate compositions, particularly treatment forms comprising compositions that are bioequivalent to the currently marketed dosage forms.

### SUMMARY

In one embodiment, a fenofibrate composition comprises fenofibrate nanoparticles having an effective average particle size of less than 2000 nm, wherein the composition comprises a particle sequestrant.

In another embodiment, a fenofibrate composition comprises fenofibrate nanoparticles having an effective average particle size of less than 2000 nm, wherein the composition comprises a particle sequestrant, wherein the composition exhibits a ratio of a logarithmic transformed geometric mean AUC_{0-∞} of the composition administered in a non-fasted state to a logarithmic transformed geometric mean AUC_{0-∞} of the composition administered in a fasted state of within about 0.80 to about 1.25, and a ratio of a logarithmic transformed geometric mean Cₘₐₓ of the composition administered in a non-fasted state to a logarithmic transformed geometric mean Cₘₐₓ of the composition administered in a fasted state of within about 0.80 to about 1.25.

In yet another embodiment, a fenofibrate composition comprises fenofibrate nanoparticles having an effective average particle size of less than 2000 nm, wherein the composition comprises a particle sequestrant, and wherein the composition has less than a 25% difference in both the AUC_{0-∞}, and the Cₘₐₓ when measured under fasted compared to non-fasted conditions.

In another embodiment, a composition comprises fenofibrate nanoparticles having an effective average particle size of less than 2000 nm, wherein the composition comprises a particle sequestrant, and wherein the AUC_{0-∞}T is within a lower confidence interval limit of 80% and an upper confidence interval limit of 125% of 144652 hr*ng/ml, the AUC_{0-∞}INF is within a lower confidence interval limit of 80% and an upper confidence interval limit of 125% of 167445 hr*ng/ml, and the Cₘₐₓ is within a lower confidence interval limit of 80% and an upper confidence interval limit of 125% of 10485 ng/ml.

In one aspect, a fenofibrate composition comprises fenofibrate nanoparticles having an effective average particle size of less than 2000 nm, wherein the composition comprises no added surfactants, phospholipids, or a combination thereof, and wherein the composition exhibits a ratio of a logarithmic transformed geometric mean AUC_{0-∞} of the composition to a logarithmic transformed geometric mean AUC_{0-∞} of a reference drug of within about 0.80 to about 1.25 and a ratio of a logarithmic transformed geometric mean Cₘₐₓ of the composition to a logarithmic transformed geometric mean Cₘₐₓ of a reference drug of within about 0.80 to about 1.25; wherein the reference drug is the reference drug product of NDA #021656.

In another aspect, a fenofibrate composition comprises fenofibrate nanoparticles having an effective average particle size of less than 2000 nm, wherein the composition comprises no added surfactants, phospholipids, or a combination thereof, and wherein the composition comprises a particle sequestrant, wherein the composition exhibits a ratio of a logarithmic transformed geometric mean AUC_{0-∞} of the composition administered in a non-fasted state to a logarithmic transformed geometric mean AUC_{0-∞} of the composition administered in a fasted state of within about 0.80 to about 1.25, and a ratio of a logarithmic transformed geometric mean Cₘₐₓ of the composition administered in a non-fasted state to a logarithmic transformed geometric mean Cₘₐₓ of the composition administered in a fasted state of within about 0.80 to about 1.25.

In another aspect, a fenofibrate composition comprises fenofibrate nanoparticles having an effective average particle size of less than 2000 nm, wherein the composition comprises no added surfactants, phospholipids, or a combination thereof, and wherein the composition has less than a 25% difference in AUC_{0-∞}, and Cₘₐₓ when measured under fasted compared to non-fasted conditions.

In yet another aspect, a fenofibrate composition comprises fenofibrate nanoparticles having an effective average particle size of less than 2000 nm, wherein the composition comprises no added surfactants, phospholipids, or a combination thereof, and wherein the AUC_{0-∞} is within a lower confidence interval limit of 80% and an upper confidence interval limit of 125% of 144652 hr*ng/ml, the AUC_{0-∞} is within a lower confidence interval limit of 80% and an upper confidence interval limit of 125% of 167445 hr*ng/ml, and the Cₘₐₓ is within a lower confidence interval limit of 80% and an upper confidence interval limit of 125% of 10485 ng/ml.

In another embodiment, an active agent composition comprises active agent particles having an effective average particle size of less than 2000 nm, wherein the active agent nanoparticles and a particle sequestrant are disposed on an inert core particle, and wherein the particle sequestrant is a pH-sensitive copolymer having both hydrophobic (meth)acrylate units and acid-soluble (meth)acrylate units.

In another embodiment, an active agent composition comprises active agent nanoparticles having an effective average particle size of less than 2000 nm, wherein the active agent nanoparticles and a particle sequestrant are disposed on an inert core particle, wherein the composition comprises no surfactants or phospholipids, and wherein the active agent composition redisperses in a biorelevant medium.

In another embodiment, an active agent composition comprises active agent nanoparticles having an effective average particle size of less than 2000 nm, wherein the active agent nanoparticles and a particle sequestrant are disposed on an inert core particle, and wherein the particle sequestrant is a pH-sensitive copolymer having both hydrophobic (meth)acrylate units and acid-soluble (meth)acrylate units, wherein the composition is bioequivalent under fasted and non-fasted conditions, wherein the composition exhibits a ratio of a logarithmic transformed geometric mean AUC_{0-∞} of the composition administered in a non-fasted state to a logarithmic transformed geometric mean AUC_{0-∞} of the composition administered in a fasted state of within about 0.80 to about 1.25, and a ratio of a logarithmic transformed geometric mean Cₘₐₓ of the composition administered in a non-fasted state to a logarithmic transformed geometric mean Cₘₐₓ of the composition administered in a fasted state of within about 0.80 to about 1.25.

In another embodiment, a method of improving the bioavailability of an active agent comprises administering an active agent dosage form, the active agent dosage form comprising active agent nanoparticles having an effective average particle size of less than 2000 nm, wherein the active agent nanoparticles and a particle sequestrant are disposed on an inert core particle, wherein the composition comprises no surfactants or phospholipids, and wherein the active agent composition redisperses in a biorelevant medium.

These and other embodiments, advantages and features of the present invention are illustrated by the Figures, Detailed Description, and Examples that follow.

### BRIEF DESCRIPTION OF THE FIGURES

FIGS. 1-11 are individual plots of plasma concentration versus time for individual subjects.

FIG. 12 shows the linear squared mean average plasma concentration versus time for all 11 patients compared to TriCor®.

FIG. 13 is a flow chart showing a method of producing fenofibrate tablets.

FIG. 14 shows the particle size distribution of a fenofibrate suspension at an initial time point, shortly after milling.

FIG. 15 shows the particle size distribution of a fenofibrate suspension at 3 days at room temperature.

FIG. 16 shows the particle size distribution of a fenofibrate suspension at 7 days at room temperature.

FIG. 17 shows the particle size distribution of a fenofibrate suspension at 12 days at room temperature.

### DETAILED DESCRIPTION

Disclosed herein are compositions and methods for novel fenofibrate dosage forms, which are also applicable to other substantially water-insoluble active agents. The oral dosage forms are based on nanoparticulate active agents. In some embodiments, the nanoparticulate active agents are in combination with a particle sequestrant, which provides redispersibility of the active agent after dosing. In one embodiment, the dosage form is in a treatment form that comprises fenofibrate or fenofibric acid, and that is bioequivalent to commercially available nanoparticulate fenofibrate tablet formulations.

An “active agent” means a compound, element, or mixture that when administered to a patient, alone or in combination with another compound, element, or mixture, confers, directly or indirectly, a physiological effect on the patient. The indirect physiological effect can occur via a metabolite or other indirect mechanism. When the active agent is a compound, then salts, solvates (including hydrates) of the free compound or salt, crystalline forms, non-crystalline forms, and any polymorphs of the compound are contemplated herein. Compounds can contain one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In addition, compounds with carbon-carbon double bonds can occur in Z- and E-forms, with all isomeric forms of the compounds. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

In one embodiment, the active agent is a substantially water insoluble active agent such as, for example, fenofibrate, oxcarbazepine, metaxalone, acetyl digoxin, acyclovir analogs, albendazole, albendazole sulfoxide, alfaxalone, alprazolam, alprostadil, altretamine, amiloride, amiodarone, aminofostin, amlodipine besylate, anipamil, antithrombin III, aprepitant, atazanavir sulfate, atenolol, acetylsalicylate; atorvastatin calcium, azithromycine, azidothymidine, atovaquone, bexarotene, beclobrate, beclomethasone, belomycin, benzafibrate, benzocaine and derivatives, beta carotene, beta endorphin, beta interferon, bezafibrate, bicalutamide, binovum, biperiden, bosentan, brimonidine, bromazepam, bromocryptine, bucindolol, buflomedil, bupivacaine, busulfan, ampothecin, benztropine mesylate, bupropion, cadralazine, camptothesin, candesartan, canthaxanthin, captopril, carbamazepine, carboprost, cefalexin, cefalotin, cefamandole, cefazedone, cefdinir, cefluoroxime, cefinenoxime, cefoperazone, cefotaxime, cefoxitin, cefsulodin, ceftizoxime, chlorambucil, chromoglycinic acid, ciclonicate, ciglitazone, cilostazol, ciprofloxacine, citalopram, clarithromycin, clonidine, clopidogrel bisulfate, colesevelam hydrochloride, cortexolone, corticosterone, cortisol, cortisone, cyclosporin A and other cyclosporins, cyclophosphamide, cytarabine, cabergoline, cerivastatin, chlorpromazine, cisapride, yclobenzaprine, cyproheptadine, ceftazidime, cefuroxime, duloxetine, desocryptin, desogestrel, dexamethasone esters such as the acetate, dezocine, diazepam, diclofenac, dideoxyadenosine, dideoxyinosine, digitoxin, digoxin, dihydroergotamine, dihydroergotoxin, diltiazem, dopamine antagonists, doxorubicin, delavirdine, desmopressin, dipyridamole, dolasetron, dacarbazine, econazole, endralazine, enkephalin, enalapril, epoprostenol, estradiol, estramustine, etofibrate, etoposide, enalapril maleate, enalaprilat, factor ix, factor viii, felbamate, fenbendazole, fexofenadine HCl, finasteride, flunarizin, flurbiprofen, 5-fluorouracil, flurazepam, fosfomycin, fosmidomycin, furosemide, famotidine, felodipine, furazolidone, fluconazole, gallopamil, gamma interferon, ganciclovir, gentamicin, gepefrine, gliclazide, glimepiride, glipizide, glyburide, griseofulvin, haptoglobulin, hepatitis B vaccine, hydralazine, hydrochlorothiazide, hydrocortisone, ibuprofen, ibuproxam, indinavir, indomethacin, iodinated aromatic x-ray contrast agents such as iodamide, ipratropium bromide, Itraconazole, ketoconazole, ketoprofen, ketotifen, ketotifen fumarate, K-strophanthin, irbesartan, lamotrigine, latanoprost, labetalol, lactobacillus vaccine, letrozole, lidocaine, idoflazin, lisuride, lisuride hydrogen maleate, lopinavir, lorazepam, lovastatin, lansoprazole, loratadine, loxapine, mefloquine, mefenamic acid, meloxicam, melphalan, memantine, mercaptopurine, mesulergin, metergoline, methotrexate, methyl digoxin, methylprednisolone, metronidazole, metisoprenol, metipranolol, metkephamide, metolazone, metoprolol, metoprolol tartrate, miconazole, miconazole nitrate, milrinone lactate, minoxidil, misonidazol, mirtazapine, molsidomin, mebendazole, minocycline, mitoxantrone, mycophenolate, nadolol, nafiverine, nafazatrom, naproxen, nateglinide, natural insulins, navelbine, nesapidil, nicardipine, nicorandil, nifedipine, niludipin, nimodipine, nitrazepam, nitrendipine, nitrocamptothesin, 9-nitrocamptothesin, nelfinavir mesylate, norfioxacin, olanzapine, olmesartan, oxazepam, oxprenolol, oxytetracycline, omeprazole, paclitaxel, penclomedine, pioglitazone, penicillins such as penicillin G benethamine, penecillin O, phenylbutazone, picotamide, pindolol, piposulfan, piretanide, piribedil, piroxicam, pirprofen, plasminogenici activator, prednisolone pregnenolone, procarbacin, procaterol, progesterone, proguanil, proinsulin, propafenone, propanolol, propentofyllin, propofol, propranolol, penciclovir, pimozide, quazepam, rifabutin, rifapentin, riluzole, risperidone, ritonavir, rofecoxib, rosiglitazone, raloxifene, rifampin, risperidone, rizatriptan, saquinavir, sildenafil, acetyl-sulfisoxazole semi-synthetic insulins, sertraline, simvastatin, sirolimus, sobrerol, somastotine and its derivatives, somatropin, stilamine, sulfinalol hydrochloride, sulfinpyrazone, suloctidil, suprofen, sulproston, synthetic insulins, tacrolimus, tamoxifen, tamsulosin HCl, talinolol, taxol, taxotere, temazepam, teniposide, terbinafine HCl, testosterone, testosterone propionate, testosterone undecanoate, tetracane HI, thalidomide, thiabendazole, thioguanine, tiaramide HCl, tolmetin, trandolapril, tranilast, triamterene, trimetrexate, triquilar, troglitazone, tromantadine HCl, trovafloxacin, urokinase, valdecoxib, valium, valproic acid and valproex, verapamil, vidarabine and vidarabine phosphate sodium salt, vinblastine sulfate, vinburin, vincamine, vincristine, vindesine, vinpocetine, vitamin A and its derivatives (retinoic acid, isotretinoin, etc.), vitamin E succinate, x-ray contrast agents, zafirlukast, zaleplon, zolpidem, and combinations comprising one or more of the foregoing active agents.

In a specific embodiment, the active agent is fenofibrate, i.e., the 1-methyl ethyl ester of fenofibric acid. Fenofibrate is known to be metabolized in the body to fenofibric acid, its active metabolite. Thus, after the oral administration of fenofibrate, fenofibric acid is found in plasma. In another specific embodiment, the active agent is fenofibric acid.

By “substantially water-insoluble” or “poorly soluble” active agent, it is meant an agent having a water solubility of less than 1 mg/ml.

“Efficacy” means the ability of an active agent administered to a patient to produce a therapeutic effect in the patient.

“Safety” means the incidence or severity of adverse events associated with administration of an active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, or concomitant medication).

A “dosage form” means a unit of administration of an active agent. Examples of dosage forms include tablets, capsules, injections, suspensions, liquids, emulsions, creams, ointments, suppositories, inhalable forms, transdermal forms, and the like. A “treatment form” refers to a dosage form of fenofibric acid or fenofibrate that is bioequivalent to current commercially available oral fenofibrate formulations.

In one embodiment, a “treatment form” refers to a dosage form of fenofibrate that is bioequivalent to Abbott Laboratories' TriCor® as presently marketed.

“Bioavailability” means the extent or rate at which an active agent is absorbed into a living system or is made available at the site of physiological activity. For active agents that are intended to be absorbed into the bloodstream, bioavailability data for a given formulation can provide an estimate of the relative fraction of the administered dose that is absorbed into the systemic circulation. “Bioavailability” can be characterized by one or more pharmacokinetic parameters.

“Pharmacokinetic parameters” describe the in vivo characteristics of an active agent (or surrogate marker for the active agent) over time, such as plasma concentration (C), Cₘₐₓ, Cₙ, C₂₄, Tₘₐₓ, and AUC. “Cₘₐₓ” is the measured concentration of the active agent in the plasma at the point of maximum concentration. “Cₙ” is the measured concentration of an active agent in the plasma at about n hours after administration. “C₂₄” is the measured concentration of an active agent in the plasma at about 24 hours after administration. The term “Tₘₐₓ” refers to the time at which the measured concentration of an active agent in the plasma is the highest after administration of the active agent. “AUC” is the area under the curve of a graph of the measured concentration of an active agent (typically plasma concentration) vs. time, measured from one time point to another time point. For example AUC₀₋ₜ is the area under the curve of plasma concentration versus time from time 0 to time t. The AUC_{0-∞} or AUC_{0-INF} is the calculated area under the curve of plasma concentration versus time from time 0 to time infinity.

Food is typically a solid food with sufficient bulk and fat content that it is not rapidly dissolved and absorbed in the stomach. In one embodiment, “food” is a meal, such as breakfast, lunch, or dinner. The terms “taken with food”, “fed” and “non-fasted” are equivalent and are as given by FDA guidelines and criteria. In one embodiment, “with food” means that the dosage form is administered to a patient between about 30 minutes prior to about 2 hours after eating a meal. In another embodiment, “with food” means that the dosage is administered at substantially the same time as the eating the meal.

The terms “without food,” “fasted,” and “an empty stomach” are equivalent and are as given by FDA guidelines and criteria. In one embodiment, “fasted” means the condition of not having consumed solid food for at least about 1 hour prior or at least about 2 hours after such consumption. In another embodiment, “fasted” means the condition of not having consumed solid food for at least about 1 hour prior to at least about 2 hours after such consumption.

For the purposes of biostudy and the determination of bioequivalence, a “fasted patient” means a patient who does not eat any food, i.e., fasts, for at least 10 hours before the administration of a dosage form of active agent and who does not eat any food and continues to fast for at least 4 hours after the administration of the dosage form. The dosage form is administered with 240 ml of water during the fasting period, and water can be allowed ad libitum after 2 hours.

For the purposes of biostudy and the determination of bioequivalence, a “non-fasted patient” means a patient who fasts for at least 10 hours overnight and then consumes an entire test meal within 30 minutes of first ingestion. The dosage form is administered with 240 mL of water at 30 minutes after first ingestion of the meal. No food is then allowed for at least 4 hours post-dose. Water can be allowed ad libitum after 2 hours. A high fat test meal provides approximately 1000 calories to the patient of which approximately 50% of the caloric content is derived from fat content of the meal. A representative high fat high calorie test meal comprises 2 eggs fried in butter, 2 strips of bacon, 2 slices of toast with butter, 4 ounces of hash brown potatoes, and 8 ounces of whole milk to provide 150 protein calories, 250 carbohydrate calories, and 500 to 600 fat calories.

In one aspect, the present invention relates to oral fenofibrate or fenofibric acid treatment forms that are bioequivalent to commercially available nanoparticulate tablet formulations. TriCor® 145 and 48 were approved by the FDA under NDA #021656 on Nov. 5, 2004. The approved prescribing information for TriCor® 145 and 48 states that “Exposure to fenofibric acid in plasma, as measured by Cₘₐₓ and AUC, is not significantly different when a single 145 mg dose of fenofibrate is administered under fasted or non-fasted conditions.”

Under U.S. FDA guidelines, two products (e.g., an inventive composition and TriCor® 145) or methods (e.g., dosing under non-fasted versus fasted conditions) are bioequivalent if the 90% Confidence Intervals (CI) for the ratios of a log transformed geometric mean of AUC_{0-∞} for the first product or method compared to the second product or method, and Cₘₐₓ for the first product or method compared to the second product or method, are within 0.80 to 1.25 (Tₘₐₓ measurements are not relevant to bioequivalence for regulatory purposes). To show bioequivalency between two compositions or methods pursuant to Europe's EMEA guidelines, the 90% CI for the ratios of a log transformed geometric mean of AUC_{0-∞} for the first product or method compared to the second, must be within 0.80 to 1.25 and the 90% CI for the ratios of a log transformed geometric mean of Cₘₐₓ for the first product or method compared to the second must be within 0.70 to 1.43.

Thus, in one embodiment, the oral fenofibrate or fenofibric acid treatment form is bioequivalent to TriCor® 145 mg or 48 mg. In another embodiment, the oral fenofibrate or fenofibric acid treatment form is bioequivalent to a reference drug wherein the reference drug is 145 or 48 mg fenofibrate formulations comprising nanoparticles of fenofibrate having associated with the surface thereof a surface stabilizer comprising hypromellose, sodium lauryl sulfate and dioctyl sodium sulfosuccinate.

Bioequivalency can be established by a number of criteria, for example 90% Confidence Intervals of 0.80 to 1.25 for a log transformed geometric mean of AUC_{0-∞}, and Cₘₐₓ. Accordingly, in a given experiment, the oral fenofibrate or fenofibric acid treatment form can be considered to be “bioequivalent” to the reference TriCor® 145 or 48 of NDA #021656 if both of the obtained ln-transformed geometric mean Test/Reference AUC_{inf} and Cₘₐₓ ratio percents along with their corresponding lower and upper CI limits are within a lower limit of 80% and an upper limit of 125%. The water insolubility of fenofibrate can lead to substantial inter-experiment variability in the pharmacokinetic parameters measured for fenofibrate. Thus, for direct comparison between a fenofibrate treatment form and TriCor® 145 or 48, it is sometimes preferred to determine the pharmacokinetic parameters for the fenofibrate treatment form and TriCor® 145 or 48 side-by-side in the same set of experiments.

In a specific embodiment, the oral fenofibrate or fenofibric acid treatment form has substantially the same AUC₀₋ₜ, AUC_{0-∞}, and Cₘₐₓ as TriCor® 145, wherein the AUC_{0-∞}T of TriCor® 145 is, within a lower confidence interval limit of 80% and an upper confidence interval limit of 125%, measured as 144652 hr*ng/ml, the AUC_{0-∞} of TriCor® 145 is, within a lower confidence interval limit of 80% and an upper confidence interval limit of 125%, measured as 167445 hr*ng/ml, and the Cₘₐₓ of TriCor® 145 is, within a lower confidence interval limit of 80% and an upper confidence interval limit of 125%, measured as 10485 ng/ml.

In another specific embodiment, the oral fenofibrate or fenofibric acid treatment form has substantially the same AUC₀₋ₜ, AUC_{0-∞}, and Cₘₐₓ of TriCor® 145, wherein the AUC_{0-∞}T of TriCor® 145 is measured as 120768 to 156764 hr*ng/ml, the AUC_{0-∞} of TriCor® 145 is measured as 139040 to 186493 hr*ng/ml, and the Cₘₐₓ, of TriCor® 145 is measured as 9096 to 11393 ng/ml.

The invention also encompasses oral fenofibrate or fenofibric acid dosage forms having reduced non-fasting/fasting effects compared to prior formulations such as, for example TriCor® 160 mg or 54 mg. For TriCor® 160 mg and 54 mg, the absorption of fenofibrate is reportedly increased by about 35% when administered with food. Thus, in this embodiment, the difference in pharmacokinetic parameters between the fed and fasted state is less than 35%, specifically less than 25%, more specifically less than 10%.

In order to obtain bioequivalency, the oral compositions contain active agent nanoparticles, e.g., fenofibrate nanoparticles, that have an average particle size of less than about 2000 nm (i.e., 2 microns), less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, or less than 400 nm, as measured by light-scattering methods, microscopy, or other appropriate methods. As used throughout this specification, “particle size” refers to the largest diameter (i.e., dimension) of the particle.

More specifically, in order to obtain bioequivalency, the oral compositions contain active agent nanoparticles, e.g., fenofibrate nanoparticles, that have an effective average particle size of less than about 2000 nm (i.e., 2 microns), less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, or less than 400 nm, as measured by light-scattering methods, microscopy, or other appropriate methods. By “an effective average particle size of less than about 2000 nm” it is meant that at least 50% of the active agent particles, (e.g., fenofibrate particles) have a particle size of less than the average, by weight, i.e., less than about 2000 nm, 1900 nm, 1800 nm, etc., when measured by the above-noted techniques. Preferably, at least about 70%, about 90%, or about 95% of the particles have a particle size of less than the effective average, i.e., less than about 2000 nm, 1900 nm, 1800 nm, 1700 nm, etc. As is understood in the art, the value for D₅₀ of a nanoparticulate active agent is the particle size below which 50% of the particles fall, by weight. Similarly, D₉₀ is the particle size below which 90% of the fibrate particles fall, by weight. In certain embodiments, average diameter is used interchangeably with average particle size.

The nanoparticulate active agents can further have a narrow particle size distribution. In particular, less than 25%, less than 15%, less than 10%, or less than 5% (by weight) of the particles have a particle size greater than 4 micrometers.

In another embodiment, less than 25%, less than 15%, less than 10%, or less than 5% (by weight) of the particles have a particle size greater than 3 micrometers. In still another embodiment, less than 25%, less than 15%, less than 10%, or less than 5% (by weight) of the particles have a particle size greater than 2 micrometers. In another embodiment, less than 50%, less than 35%, less than 20%, or less than 10% (by weight) of the particles have a particle size greater than 1 micrometer. In another embodiment, less than 50%, less than 35%, less than 20%, or less than 10% (by weight) of the particles have a particle size greater than 0.5 micrometers.

Further in order to obtain bioequivalency and/or redispersibility, the active agent composition comprises active agent nanoparticles as described above and a compound that sequesters the nanoparticles during at least a portion of the processing to form the compositions, dosage forms and treatment forms, i.e., a sequestering agent or “particle sequestrant.” The particle sequestrant provides, among other advantages, improved bioavailability of the poorly-water soluble active agent. Without being bound by theory, it is hypothesized that during formulation, the particle sequestrant isolates the nanoparticulate active agents from adjacent nanoparticles. Agglomeration and/or crystal growth of the particles during formulation is accordingly inhibited, so that nanoparticles (rather than larger particles) are provided to the body upon dissolution (or other type of delivery) of the dosage form. It is also possible that the particle sequestrant inhibits agglomeration and/or crystal growth of the poorly water-soluble nanoparticulate active agents during or immediately after dissolution or other delivery in the body.

It has been found that effective particle sequestrants include pH-sensitive copolymers having both hydrophobic (meth)acrylate units and acid-soluble (meth)acrylate units. As used herein, (meth)acrylate encompasses both acrylates and methacrylates. Hydrophobic (meth)acrylate units are derived from (meth)acrylate monomers having a water solubility of less than or equal to 2 g per 100 g of water, measured at 25° C., specifically less than or equal to 1.5 g, more specifically less than or equal to 1.0 g. Acid-soluble (meth)acrylate units are derived from monomers containing basic groups, for example amines, and impart solubility and/or swellability to the polymer when in aqueous media having a pH of less than 5.5, specifically less than 5.0, more specifically less than 4.5, and even more specifically less than 4.0. In one embodiment the pH sensitive copolymer solubilizes or swells at a pH of about 3, as found in the stomach, but remains insoluble or deswelled at pH's greater than 4. Other types of units can be present in the polymer, provided that such units do not substantially adversely impact the sequestering activity of the polymer.

Exemplary (meth)acrylate monomers having a water solubility of 2 g or less per 100 g of water, measured at 25° C. include the C₁₋₁₈ hydrocarbyl esters of (meth)acrylic acid. “Hydrocarbyl” as used herein includes alkyl, cycloalkyl, alkylaryl, arylalkyl, and aryl groups that are unsubstituted or substituted with up to two heteroatoms, including halogen (fluorine, chlorine, bromine and iodine), nitrogen, oxygen, and sulfur. It is to be understood that any substituent (e.g., a hydroxy group) that increases the solubility of the monomer to above 2 g/100 g of water is not within the scope of the present compounds. Specific exemplary C₁₋₁₂ hydrocarbyl esters include methyl (meth)acrylate, ethyl (meth)acrylate, n-propyl (meth)acrylate, 2-propyl (meth)acrylate, cyclohexyl (meth)acrylate, dodecyl (meth)acrylate, 2-ethylhexyl (meth)acrylate, octyl (meth)acrylate, t-butyl (meth)acrylate n-butyl (meth)acrylate, phenyl (meth)acrylate, butyl (meth)acrylate, methyl methacrylate, benzyl (meth)acrylate, phenyl (meth)acrylate, and propyl methacrylate. Specific monomers are t-butyl (meth)acrylate, methyl methacrylate, and n-butyl (meth)acrylate.

In one embodiment, a combination of hydrophobic (meth)acrylate monomers is used. A specific combination comprises a hydrophobic (meth)acrylate monomer having a solubility of 1 to 2 g/100 g of water at 20° C., and a hydrophobic (meth)acrylate monomer having a solubility of less than 1 g/100 g of water at 20° C. An exemplary combination of hydrophobic (meth)acrylate monomers is a combination of methyl (meth)acrylate and butyl (meth)acrylate. The relative molar ratio of the hydrophobic (meth)acrylate having a solubility of 1 to 2 g/100 g of water at 20° C. to hydrophobic (meth)acrylate having a solubility of less than 1 g/100 g of water at 20° C., can vary widely depending on the active agent, the formulation solvent, availability, and like considerations, and can readily be determined by one of ordinary skill in the art without undue experimentation. In general, the molar ratio of the hydrophobic (meth)acrylate having a solubility of 1 to 2 g/100 g of water at 20° C. to hydrophobic (meth)acrylate having a solubility of less than 1 g/100 g of water at 20° C. is 95:5 to 5:95, specifically 80:20 to 20:80, more specifically 70:30 to 30:70.

Exemplary (meth)acrylate monomers containing basic groups are copolymerizable with the hydrophobic (meth)acrylate monomers, and have a functional group having a pKb of less than 20, specifically less than 10, more specifically less than 5. Nitrogen-containing functional groups are preferred. Tertiary amines are particularly useful, wherein the amine is connected to the (meth)acrylate via one of the amine substituents, and each of the substituents is the same or different. Exemplary substituents include C₁₋₁₂ hydrocarbyl groups, specifically unsubstituted C₁₋₁₂ hydrocarbyl groups, and even more specifically unsubstituted C₁₋₁₂ alkyl or cycloalkyl groups.

Exemplary (meth)acrylate monomers containing basic groups include 2-dimethylamino methyl (meth)acrylate, 2-dimethylamino ethyl (meth)acrylate, 2-diethylamino ethyl (meth)acrylate, 2-piperidinyl ethyl (meth)acrylate, and 2-(di-tert-butylamino) ethyl (meth)acrylate, specifically 2-dimethylamino ethyl methacrylate and 2-diethylamino ethyl acrylate.

The relative molar ratios of the hydrophobic (meth)acrylate and (meth)acrylate containing a basic group can vary widely depending on the active agent, the formulation solvent, availability, and like considerations, and can readily be determined by one of ordinary skill in the art without undue experimentation. In general, the molar ratio of the hydrophobic (meth)acrylate and (meth)acrylate containing a basic group is 95:5 to 5:95, specifically 80:20 to 20:80, more specifically 70:30 to 50:50. The copolymer can have a molecular weight of 10,000 to 800,000, specifically 50,000 to 500,000.

A specific particle sequestrant is a butyl methacrylate-(2-dimethylaminoethyl methacrylate)-methyl methacrylate copolymer (1:2:1) available in granular form under the trade name EUDRAGIT® E-100. This copolymer has a mean molecular weight of 150,000, a viscosity of 3-12 mPas at 20° C., a refractive index of N²⁰_{D}: 1.380-1.385 and a relative density of d²⁰₄: 0.810-0.820. The same polymer is available in powder form under the trade name EUDRAGIT® E PO. In one embodiment, the particle sequestrant consists essentially of a butyl methacrylate-(2-dimethylaminoethyl methacrylate)-methyl methacrylate copolymer (1:2:1), for example the copolymer having a mean molecular weight of 150,000, a viscosity of 3-12 mPas at 20° C., a refractive index of N²⁰_{D}: 1.380-1.385 and a relative density of d²⁰₄: 0.810-0.820. In another embodiment, the particle sequestrant consists of butyl methacrylate-(2-dimethylaminoethyl methacrylate)-methyl methacrylate copolymer (1:2:1), for example the copolymer having a mean molecular weight of 150,000, a viscosity of 3-12 mPas at 20° C., a refractive index of N²⁰_{D}: 1.380-1.385 and a relative density of d²⁰₄: 0.810-0.820.

The particle sequestrant and the nanoparticulate active agent can be formulated using a variety of methods to provide the desired bioequivalency. In one embodiment, the particle sequestrant and the bioactive agent are combined and processed using standard techniques for tablet, capsule, suspension, or liquid formulation. The relative ratio of active agent and particle sequestrant will vary depending on the particular active agent and particle sequestrant used, the size of the nanoparticles, the other components in the formulation, and like considerations. Generally the weight ratio of active agent to particle sequestrant is 99:1 to 50:50, specifically 95:5 more specifically 90:10.

In a variation of this embodiment, the fenofibrate nanoparticles contain no added surfactants. In another embodiment, the fenofibrate formulation comprises no added surfactant. As used herein, a surfactant is limited to amphipathic compounds (as opposed to polymers) that contain both a hydrophobic region and a hydrophilic region. Surfactants can be anionic, cationic, zwitterionic, or nonionic. Specific surfactants that are excluded from the scope of the composition in this embodiment are sodium lauryl sulfate, sodium dioctyl sulfosuccinate, and phospholipids (a class of lipids formed from a fatty acid, a phosphate group, a nitrogen-containing alcohol and a backbone such as a glycerol backbone or a sphingosine backbone).

In another embodiment, the active agent and the particle sequestrant are co-processed, then combined with an inert particle. Such a composition is referred to as a fenofibrate granulate. Accordingly, in this embodiment the active agent composition comprises fenofibrate nanoparticles having an average or effective average particle size of less than 2000 nm, a particle sequestrant, and a hydrophilic particle. The combination of the active agent and the particle sequestrant can be disposed onto the hydrophilic particle as a layer that partially or entirely covers the particle.

Exemplary inert particles are also hydrophilic, dissolving readily in the body, and include, for example, sugars such as lactose, mannitol, dextrose and sorbitol; microcrystalline cellulose; calcium phosphate; lactose; and combinations comprising one or more of the foregoing inert particles. In one embodiment, the inert particles have an average diameter of 50 to 500 μm. As used herein, “calcium phosphate” includes a variety of materials that calcium ions (Ca²⁺) together with orthophosphates (PO43−), metaphosphates, or pyrophosphates (P₂O₇⁴⁻) and optionally hydrogen, halogen ions, or hydroxide ions, for example tricalcium phosphate, dicalcium phosphate dihydrate, and dicalcium phosphate, anhydrous, available under the trade name A-Tab® from Innophos, Cranbery, N.J.

In one embodiment, the granulate comprising the co-processed active agent and the particle sequestrant combined with an inert particle is coated with a coating composition. Exemplary coating materials for the granulate include, for example, a surfactant, a water-soluble polymer, a water-insoluble polymer, or a combination comprising one or more of the foregoing coating materials. Exemplary surfactants include sodium lauryl sulfate. Exemplary water-soluble polymers include hydroxyethylcellulose, hydroxypropylcellulose, hydroxyethylmethylcellulose, hydroxypropylmethylcellulose, carboxymethylcellulose, sodium carboxymethylcellulose, polyethylene glycol, and combinations comprising one or more of the foregoing water soluble polymers. Exemplary water insoluble polymers include, for example, an acrylic polymer, an acrylic copolymer, such as a methacrylic acid-ethyl acrylate copolymer, ethyl cellulose, or a combination comprising one or more of the foregoing water insoluble polymers.

In one embodiment, an oral fenofibrate composition comprising fenofibrate nanoparticles is bioequivalent to TriCor® 145 mg or 48 mg, wherein the composition comprises a particle sequestrant.

In one embodiment, an active agent composition, e.g., a fenofibrate composition, is one in which administration of the composition to a subject in a fasted state is bioequivalent to administration of the composition to a subject in a non-fasted state. The difference in Cₘₐₓ and AUC_{0-∞} for the active agent, e.g., fenofibrate, composition, when administered in the non-fasted versus the fasted state, is less than about 35%, less than about 25%, less than about 20%, less than about 15%, less than about 10%, less than about 5%, or less than about 3%.

In another embodiment, an oral fenofibrate composition comprises no added surfactants, phospholipids, or a combination thereof. The composition optionally comprises a particle sequestrant, which is then optionally disposed on an inert core particle.

In one embodiment, an oral fenofibrate composition comprises no added surfactants, phospholipids, or a combination thereof, wherein both the ln-transformed geometric mean Test/Reference AUC_{∞} and Cₘₐₓ ratio percents along with their corresponding lower and upper confidence interval limits are within a lower limit of 80% and an upper limit of 125% when compared to the reference drug product of NDA #021656. In another embodiment, a fenofibrate composition comprises no added surfactants, phospholipids, or a combination thereof, wherein the composition is bioequivalent under fasted and non-fasted conditions, wherein bioequivalency is established by 90% Confidence Intervals of 0.80 to 1.25 for a log transformed geometric mean of AUC_{0-∞} and Cₘₐₓ. In yet another embodiment, a fenofibrate composition comprises no added surfactants, phospholipids, or a combination thereof, wherein the composition has less than a 25% difference or less than a 20% difference in AUC_{0-∞}, and Cₘₐₓ when measured under fasted compared to non-fasted conditions.

In another embodiment, an oral fenofibrate composition comprises no added surfactants, phospholipids, or a combination thereof, and has substantially the same AUC₀₋ₜ, AUC_{0-∞} and Cₘₐₓ of TriCor® 145, wherein the AUC₀₋ₜ of TriCor® 145 is, within a lower confidence interval limit of 80% and an upper confidence interval limit of 125%, measured as 144652 hr*ng/ml, the AUC_{0-∞} of TriCor® 145 is, within a lower confidence interval limit of 80% and an upper confidence interval limit of 125%, measured as 167445 hr*ng/ml, and the Cₘₐₓ of TriCor® 145 is, within a lower confidence interval limit of 80% and an upper confidence interval limit of 125%, measured as 10485 ng/ml.

In another embodiment, an oral fenofibrate composition comprises no added surfactants, phospholipids, or a combination thereof, and has substantially the same AUC_{0-∞}, AUC_{0-∞} and Cₘₐₓ of TriCor® 145, wherein the AUC_{0-∞} of TriCor® 145 is measured as 120768 to 156764 hr*ng/ml, the AUC_{0-∞} of TriCor® 145 is measured as 139040 to 186493 hr*ng/ml, and the Cₘₐₓ of TriCor® 145 is measured as 9096 to 11393 ng/ml.

The concentration of the active agent in the oral composition, e.g., fenofibrate, can be about 99.5% to about 0.001%, about 95% to about 0.1%, or about 90% to about 0.5%, by weight, based on the total combined weight of the fenofibrate and at least one particle sequestrant, not including other excipients. The concentration of the at least one particle sequestrant can be about 0.5% to about 99.999%, about 5.0% to about 99.9%, or about 10% to about 99.5%, by weight, based on the total combined dry weight of the active agent and at least one particle sequestrant, not including other excipients.

In another embodiment, as described above, the composition comprising active agent, e.g., fenofibrate, particles comprises a release-retarding material. Release-retarding materials can be hydrophilic and/or hydrophobic polymers. Release-retarding materials include, for example acrylic polymers, alkylcelluloses, shellac, zein, hydrogenated vegetable oil, hydrogenated castor oil, and combinations comprising one or more of the foregoing materials. The oral dosage form can contain about 1 wt % to about 80 wt % of the release-retarding material based on the total weight of the oral dosage form. Exemplary acrylic polymers include acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid-alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, and combinations comprising one or more of the foregoing polymers. The acrylic polymer can be a methacrylate copolymer with a low content of quaternary ammonium groups.

Exemplary alkylcelluloses include ethylcellulose. Those skilled in the art will appreciate that other cellulosic polymers, including other alkyl cellulosic polymers, can be substituted for part or all of the ethylcellulose.

Other exemplary hydrophobic materials are water-insoluble with more or less pronounced hydrophobic trends. The hydrophobic material can have a melting point of about 30° C. to about 200° C., more preferably about 45° C. to about 90° C. The hydrophobic material can include neutral or synthetic waxes, fatty alcohols (such as lauryl, myristyl, stearyl, cetyl or preferably cetostearyl alcohol), fatty acids, including fatty acid esters, fatty acid glycerides (mono-, di-, and tri-glycerides), hydrogenated fats, hydrocarbons, normal waxes, stearic acid, stearyl alcohol, hydrophobic and hydrophilic materials having hydrocarbon backbones, and combinations comprising one or more of the foregoing materials. Exemplary waxes include beeswax, glycowax, castor wax, carnauba wax and wax-like substances, e.g., material normally solid at room temperature and having a melting point of from about 30° C. to about 100° C., and combinations comprising one or more of the foregoing waxes.

In other embodiments, the release-retarding material can comprise digestible, long chain (e.g., C₈-C₅₀, preferably C₁₂-C₄₀), substituted or unsubstituted hydrocarbons, such as fatty acids, fatty alcohols, glyceryl esters of fatty acids, mineral and vegetable oils, waxes, and combinations comprising one or more of the foregoing materials. Hydrocarbons having a melting point of between about 25° C. and about 90° C. can be used. Of these long chain hydrocarbon materials, fatty (aliphatic) alcohols are preferred. The oral dosage form can contain up to about 60 wt % of at least one digestible, long chain hydrocarbon, based on the total weight of the oral dosage form.

Further, the sustained-release matrix or delayed release matrix can contain up to 60 wt % of at least one polyalkylene glycol.

Alternatively, the release-retarding material can comprise polylactic acid, polyglycolic acid, or a co-polymer of lactic and glycolic acid.

In one embodiment, in one method of manufacture, the active agent particles are reduced in size in the presence of at least one particle sequestrant. Alternatively, the active agent particles are contacted with one or more particle sequestrants after attrition. Other compounds, such as a diluent, can be added to the active agent or active agent/particle particle sequestrant composition during the size reduction process. Dispersions can be manufactured continuously or in a batch mode. A Dyno-Mill, or other suitable media mill can be used for the milling. The mill can be equipped with a temperature controlling unit to maintain the process temperature inside the milling chamber. The temperature of the suspension container can also be controlled.

In one specific embodiment, the pH-sensitive copolymer is dissolved in an aqueous solution, for example a buffered aqueous solution having a pH that is suitable to dissolve the pH-sensitive copolymer. Optionally, a C₁₋₃ alcohol is added to the solution as a wetting agent or to help dissolve the polymer. The alcohol is added in an amount effective to act as a wetting agent, e.g., 1-50% by volume of the combination of alcohol and water.

The water insoluble active agent is separately suspended in water, a mixture of 1-50 volume percent of a C₁₋₃ alcohol in water, or in a portion of the aqueous solution comprising the pH-sensitive copolymer. When the active agent is fenofibrate, about 1 to about 85 wt % of the total suspension comprises fenofibrate.

The active agent nanoparticle suspension is then dispersed onto the surface of an inert core particle, for example, by spraying in a fluid bed processor.

In another specific embodiment, the particulate fenofibrate compositions can be made by a process comprising forming an aqueous solution of the pH-sensitive copolymers having both hydrophobic (meth)acrylate units and acid-soluble (meth)acrylate units, e.g., UDRAGIT® E-100 or EUDRAGIT® E PO; forming a suspension of active agent, e.g., fenofibrate, in the aqueous solution; mixing and milling the suspension to form an active agent nanoparticulate suspension; and spraying the active agent nanoparticulate suspension over a powder bed comprising the inert cores to form granules having a suspension of the EUDRAGIT® polymer and fenofibrate dispersed on the surface of the inert cores. The fenofibrate suspension comprises fenofibrate particles with a particle size of 200-700 nm, in particular an average particle size of 200-700 nm, and even more particularly an effective average particle size of 200-700 nm. The fenofibrate particles further have a D₉₀ of not more than 1.5 micrometers. The particle size can be measured using a Malvern Mastersizer at a proper analysis mode. When a wet analysis mode is chosen, a dispersant is used.

In one embodiment, a fenofibrate nanoparticle suspension comprises an aqueous particle sequestrant solution having dispersed therein fenofibrate nanoparticles. In one embodiment, the suspension is free of any added solubilizing and/or stabilizing agents other than the particle sequestrant. In another embodiment, a fenofibrate nanoparticle suspension consists essentially of an aqueous particle sequestrant solution having dispersed therein fenofibrate nanoparticles. In another embodiment, a fenofibrate nanoparticle suspension consists of an aqueous particle sequestrant solution having dispersed therein fenofibrate nanoparticles. In one embodiment, a fenofibrate nanoparticle suspension is stable for up to two weeks after a particle size is measured. By stable it is meant that the average or effective average particle size of the fenofibrate nanoparticles changes by no more than 35% within 2 weeks of a first particle size measurement, specifically by no more than 15% within 2 weeks of a first particle size measurement. In another embodiment, the concentration of the particle sequestrant is 1% w/v to 25% w/v, specifically 3% w/v to 15% w/v and the concentration of fenofibrate is 5% w/v to 45% w/v, specifically 10% to 25% w/v.

The active agent, e.g., fenofibrate composition can be redispersible in a biorelevant media such that the average or effective average particle size of the redispersed active agent particles is less than about 2000 nm. Redispersion of the active agent particles to a substantially nanoparticulate particle size preserves the benefits afforded by formulating the active agent into a nanoparticulate particle size. This is because nanoparticulate active agent compositions typically benefit from the small particle size of the active agent; if the active agent does not redisperse into the small particle sizes upon administration, then “clumps” or agglomerated active agent particles are formed, owing to the extremely high surface free energy of the nanoparticulate system and the thermodynamic driving force to achieve an overall reduction in free energy. With the formation of such agglomerated particles, the bioavailability of the dosage form can fall well below that observed with the liquid dispersion form of the nanoparticulate active agent.

In one embodiment, nanoparticulate active agent, e.g., fenofibrate, compositions exhibit dramatic redispersion of the nanoparticulate active agent particles upon administration to a mammal, such as a human or animal. The reconstitution/redispersion is demonstrated in a biorelevant aqueous media such that the average or effective average particle size of the redispersed fenofibrate particles is less than about 2000 nanometers. Such biorelevant aqueous media are aqueous media that exhibit the ionic strength and pH, which form the basis for the biorelevance of the media. The pH and ionic strength are those that are representative of physiological conditions found in the human body. Such biorelevant aqueous media can be, for example, aqueous electrolyte solutions or aqueous solutions of any salt, acid, or base, or a combination thereof, which exhibit the desired pH and ionic strength.

Biorelevant pH is well known in the art. For example, in the stomach, the pH ranges from slightly less than 2 (but typically greater than 1) up to 4 or 5. In the small intestine the pH can range from 4 to 6, and in the colon it can range from 6 to 8. Biorelevant ionic strength is also well known in the art. Fasted state gastric fluid has an ionic strength of about 0.1 M, while fasted state intestinal fluid has an ionic strength of about 0.14 M.

Without being held to theory, it is believed that the pH and ionic strength of the biorelevant media is more critical than the specific chemical content. Accordingly, appropriate pH and ionic strength values can be obtained through numerous combinations of strong acids, strong bases, salts, single or multiple conjugate acid-base pairs (i.e., weak acids and corresponding salts of that acid), monoprotic and polyprotic electrolytes, etc.

Representative electrolyte solutions include, but are not limited to, HCl solutions, ranging in concentration from about 0.001 to about 0.1 M, and NaCl solutions, ranging in concentration from about 0.001 to about 0.1 M, and mixtures thereof. For example, electrolyte solutions can be, but are not limited to, about 0.1 M HCl or less, about 0.01 M HCl or less, about 0.001 M HCl or less, about 0.1 M NaCl or less, about 0.01 M NaCl or less, about 0.001 M NaCl or less, and mixtures thereof. Of these electrolyte solutions, 0.01 M HCl and/or 0.1 M NaCl, are most representative of fasted human physiological conditions, owing to the pH and ionic strength conditions of the proximal gastrointestinal tract.

Electrolyte concentrations of 0.001 M HCl, 0.01 M HCl, and 0.1 M HCl correspond to pH 3, pH 2, and pH 1, respectively. Thus, a 0.01 M HCl solution simulates typical acidic conditions found in the stomach. A solution of 0.1 M NaCl provides a reasonable approximation of the ionic strength conditions found throughout the body, including the gastrointestinal fluids, although concentrations higher than 0.1 M can be employed to simulate fed conditions within the human GI tract.

Exemplary solutions of salts, acids, bases or combinations thereof, which exhibit the desired pH and ionic strength, include but are not limited to phosphoric acid/phosphate salts+sodium, potassium and calcium salts of chloride, acetic acid/acetate salts+sodium, potassium and calcium salts of chloride, carbonic acid/bicarbonate salts+sodium, potassium and calcium salts of chloride, and citric acid/citrate salts+sodium, potassium and calcium salts of chloride.

In other embodiments, the active agent, e.g., fenofibrate, particles redisperse in an aqueous, biorelevant media have average dimensions of less than about 2000 nm, less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, or less than about 500 nm, as measured by light-scattering methods, microscopy, or other appropriate methods.

Solid dosage forms for oral administration include, but are not limited to, capsules, tablets, pills, powders, and granules. In such solid dosage forms, the active agent can be admixed with one or more of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, and combinations comprising one or more of the foregoing additives. For capsules, tablets, and pills, the dosage forms can also comprise buffering agents.

A method of improving the bioavailability of an active agent, comprises administering an active agent dosage form, the active agent dosage form comprising active agent nanoparticles having an average or effective average particle size of less than 2000 nm, wherein the active agent nanoparticles and a particle sequestrant are disposed on an inert core particle, and wherein the particle sequestrant is a pH-sensitive copolymer having both hydrophobic (meth)acrylate units and acid-soluble (meth)acrylate units. In one embodiment, the active agent dosage form redisperses in a biorelevant medium. In another embodiment, the active agent dosage form comprises no added surfactants or phospholipids. In yet another embodiment, the active agent dosage form comprises no added surfactant or phospholipid and redisperses in a biorelevant medium.

Fenofibrate compositions are useful in treating conditions such as hypercholesterolemia, hypertriglyceridemia, cardiovascular disorders, coronary heart disease, and peripheral vascular disease (including symptomatic carotid artery disease). The fenofibrate compositions can be used as adjunctive therapy to diet for the reduction of LDL-C, total-C, triglycerides, and Apo B in adult patients with primary hypercholesterolemia or mixed dyslipidemia (Fredrickson Types Ia and IIb). The fenofibrate compositions can also be used as adjunctive therapy to diet for treatment of adult patients with hypertriglyceridemia (Fredrickson Types IV and V hyperlipidemia). Markedly elevated levels of serum tryglycerides (e.g., >2000 mg/dL) can increase the risk of developing pancreatitis. The fenofibrate compositions can also be used for other indications where lipid-regulating agents are typically used.

Benefits of an oral dosage form which substantially eliminates the effect of food include an increase in subject convenience, thereby increasing subject compliance, as the subject does not need to ensure that they are taking a dose either with or without food. This benefit is significant, as with poor subject compliance an increase in the medical condition for which the drug is being prescribed can be observed, i.e., cardiovascular problems for poor subject compliance with fenofibrate.

The invention is further illustrated by the following non-limiting example.

### EXAMPLE 1

### Exemplary Fenofibrate Formulation

A particulate fenofibrate composition is made by a process comprising forming a solution of a pH-sensitive copolymer having both hydrophobic (meth)acrylate units and acid-soluble (meth)acrylate units, e.g., EUDRAGIT® E-100 or EUDRAGIT® E PO, in a buffered aqueous solution comprising alcohol as a wetting agent. To make the solution, 36 g of EUDRAGIT® E-100 or EUDRAGIT® E PO is dissolved in 613 g of water and 90 g of denatured ethanol containing 36 g sodium phosphate monobasic. 225 g of fenofibrate is added to the solution of pH-sensitive copolymer to form a fenofibrate suspension. The fenofibrate suspension is milled in a Dyno-Mill to produce a fenofibrate nanoparticle suspension. The fenofibrate nanoparticle suspension is sprayed over a powder bed comprising 868 g calcium phosphate particles having a diameter of 180 micrometers (A-TAB) to form granules having the EUDRAGIT® polymer and fenofibrate nanoparticles dispersed on the surface of the inert cores. Spraying is performed in a fluid bed granulator. The fenofibrate nanoparticles have an effective average particle size of 200-700 nm, specifically 300 nm, and a D₉₀ of not more than 1.5 micrometers, specifically 590 nanometers. The particle size was measured with Malvern Mastersizer S with a mixture of dispersant containing water, ethanol, EUDRAGIT® polymer, and sodium phosphate monobasic.

The overall composition is given in Table 1.

TABLE 1

| Component | Mg/tablet | Wt % |
|---|---|---|
| Fenofibrate | 145 | 8.53 |
| Sodium phosphate monobasic monohydrate | 23.3 | 1.36 |
| EUDRAGIT ® E-100 or EUDRAGIT ® E PO | 23.2 | 1.36 |
| Dibasic Calcium phosphate USP (A-tab) | 1461 | 85.9 |
| Croscarmelose sodium, Ac-Di-Sol | 40 | 2.35 |
| Magnesium stearate | 8 | 0.47 |
| Purified water | 395* | |
| Denatured alcohol | 58* | |
| | 1700 | 100 |

*Removed during process

The fenofibrate-containing granules are then blended with Ac-Di-Sol. The screened magnesium stearate is added in to the blend to form a final blend. The final blend is compressed into tablets.

### EXAMPLE 2

### Biostudy of Exemplary Fenofibrate Formulation

As used herein, for the purposes of biostudy and the determination of bioequivalence, a fasted patient is defined as a patient who does not eat any food, i.e., fasts for at least 10 hours before the administration of a dosage form of fenofibrate and who does not eat any food and continues to fast for at least 4 hours after the administration of the dosage form. The dosage form is administered with 240 ml of water during the fasting period, and water can be allowed ad libitum after 2 hours.

The study was designed as a randomized, single-dose two-way crossover to compare the pharmacokinetic parameters of the invention again that of TRICOR®. Twelve healthy adult subjects participated in this comparison study and 11 of the subjects completed the study. Subjects received two separate drug administration treatments in assigned periods, one treatment per period, according to the randomization schedule. Dosing days were separated by a washout period of at least seven days. Blood samples were drawn prior to dosing (pre-dose) and at 1, 1.5, 2, 2.5, 3, 3.5, 4, 5, 6, 7, 8, 9, 10, 11, 12, 18, 24, 36, and 48 hours post-dose. The samples were then analyzed for fenofibric acid.

The following pharmacokinetic parameters may be determined from the plasma concentration data.

The area under the plasma concentration versus time curve [AUCT] may be calculated using the linear trapezoidal rule from the zero time point to the last measured concentration.

The area under the plasma concentration versus time curve from zero to infinity [AUC_{0-INF}] may be calculated by adding Cₜ/Kₑₗₘ to AUC where Cₜ is the last measured concentration and Kₑₗₘ is the elimination rate constant.

The maximum observed plasma concentration [Cₘₐₓ] may be obtained by inspection. The Cₘₐₓ may also be designated as CMAX.

The time to maximum plasma concentration [Tₘₐₓ,] may be obtained by inspection. If the same maximum plasma concentration occurs at more than one time point, the first may be chosen as Tₘₐₓ.

The terminal elimination rate constant [Kₑₗₘ] may be obtained from the slope of the line, fitted by linear least squares regression, through the terminal points of the ln(base e) of the concentration versus time plot for these points.

The half-life [T_{1/2}] may be calculated by the equation T_{1/2}=0.693/Kₑₗₘ.

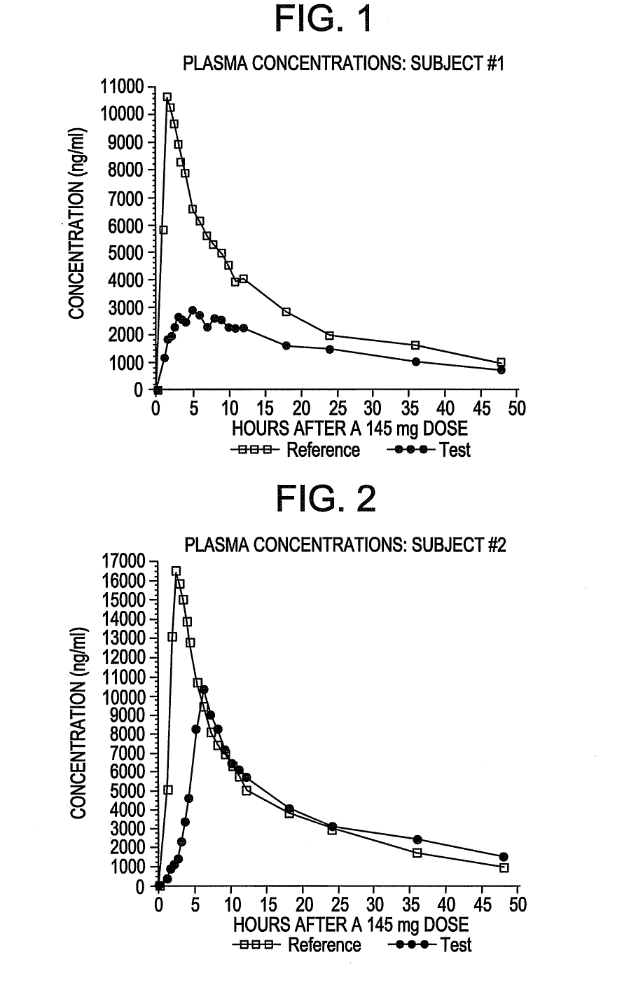

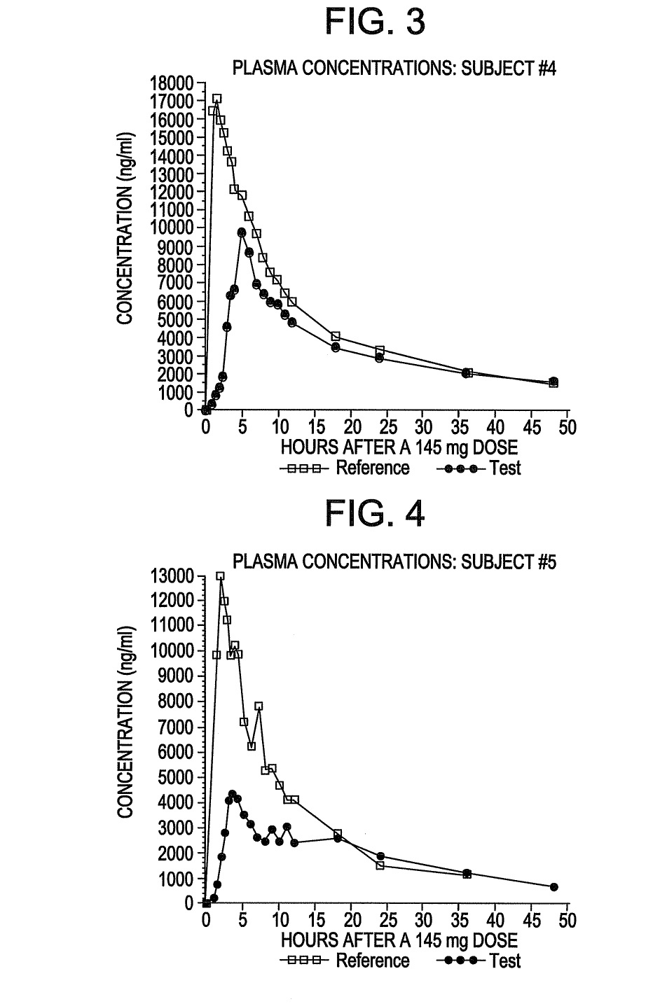

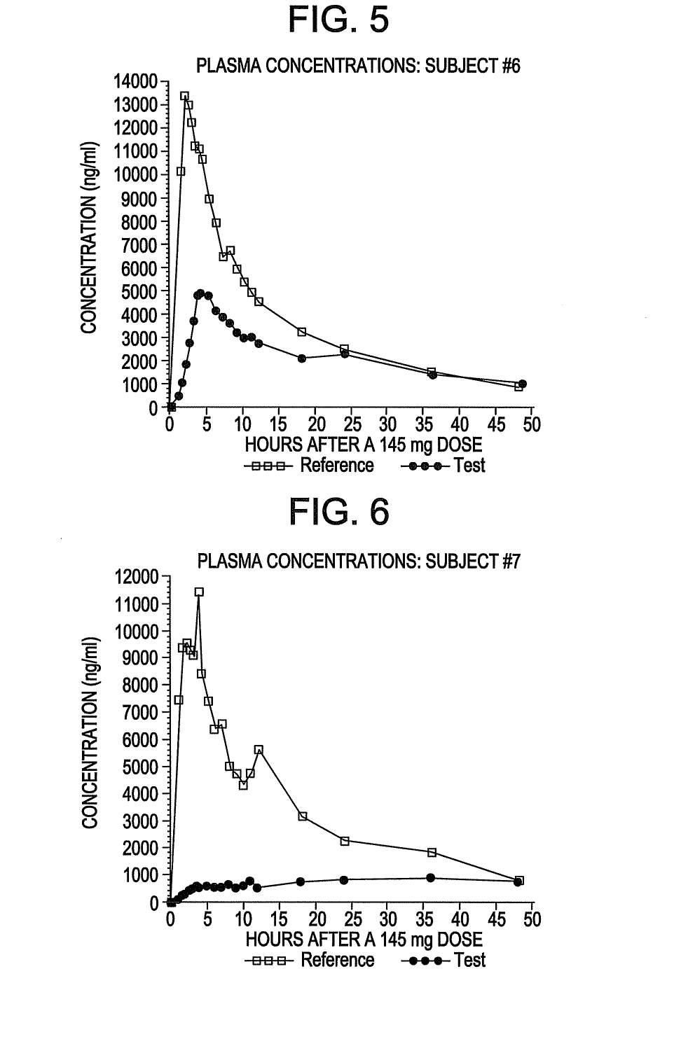

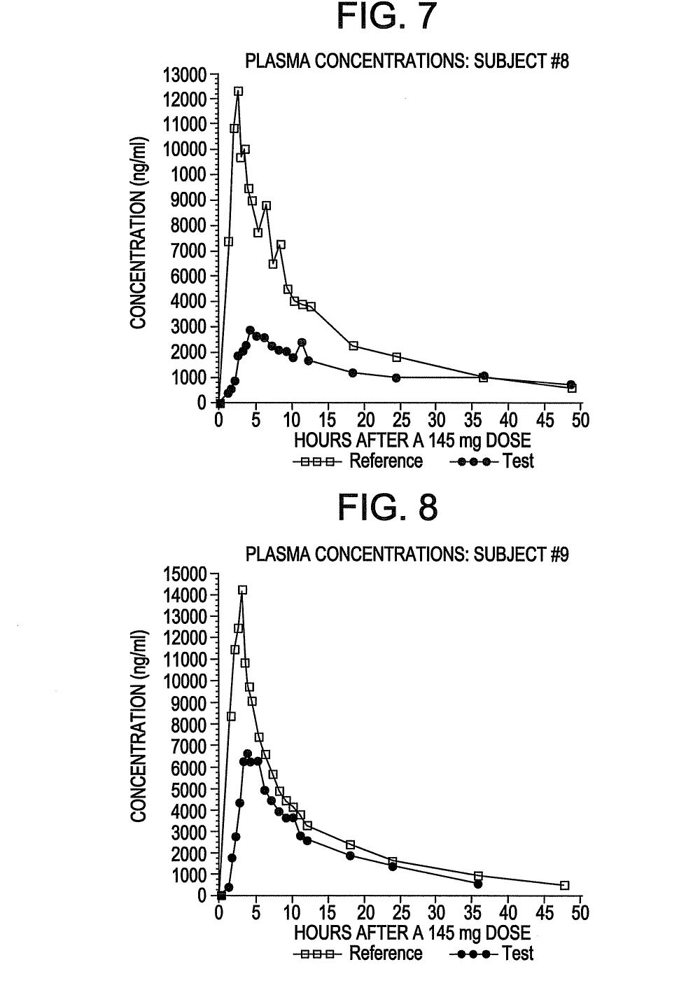

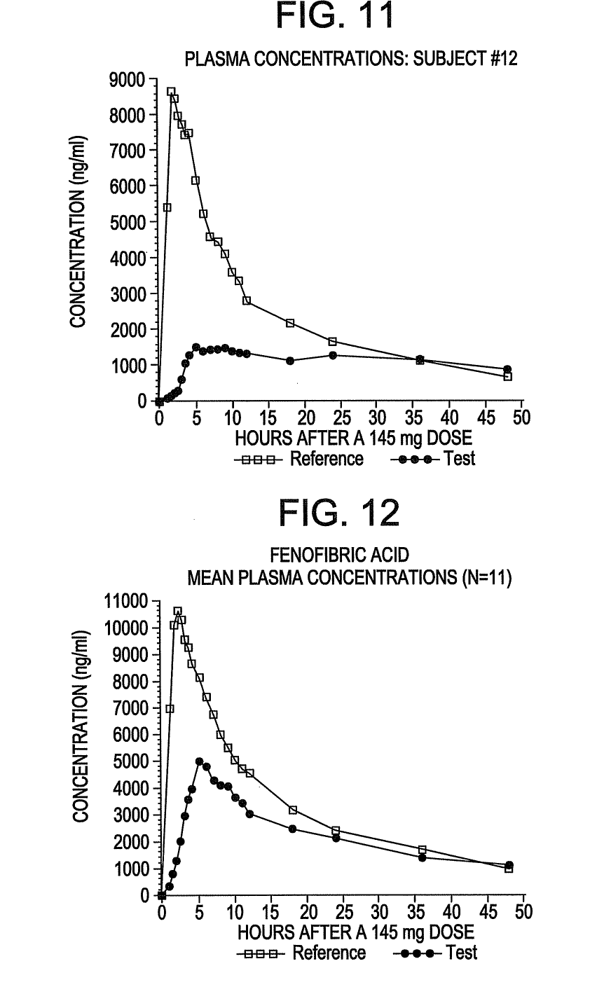

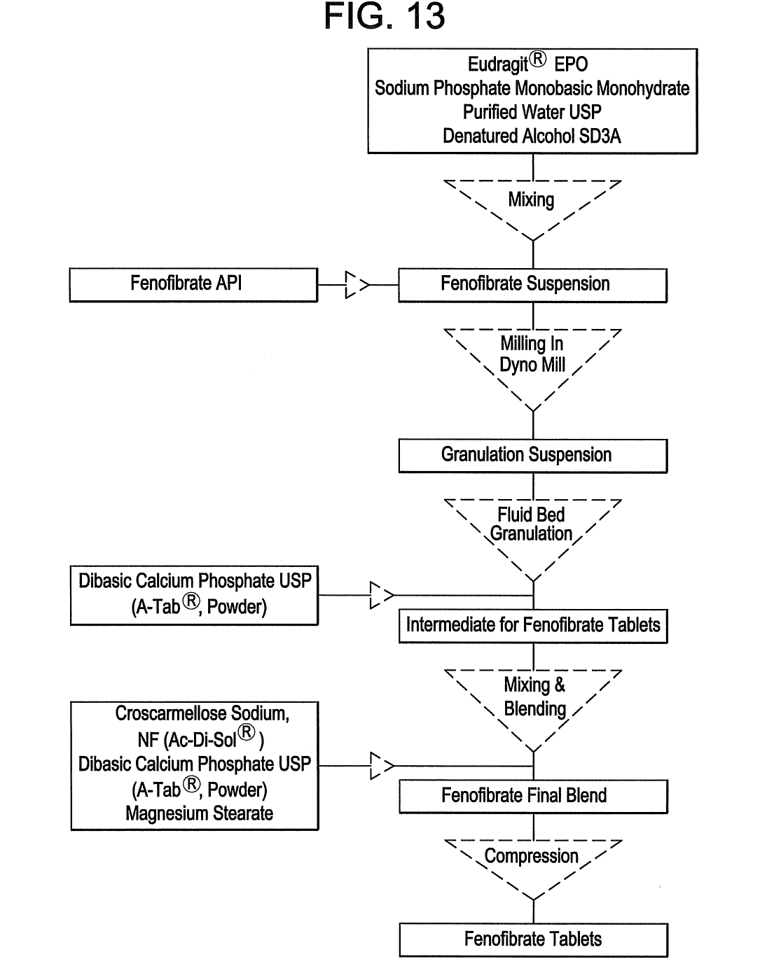

The data for 11 individual subjects is given in Table 2 and FIGS. 1-11 and the average data in Table 3 and FIG. 12.

TABLE 2
Pharmacokinetic parameters for individual human subjects

| | | AUC₀₋ₜ, | AUC_{0-∞}, | Cmax, | |
|---|---|---|---|---|---|
| Subject | Treatment | hr*ng/ml | hr*ng/ml | ng/ml | Tmax, hr |
| 1 | Invention | 72532 | 94785 | 2878 | 5 |
| | TRICOR ® | 144630 | 170426 | 10668 | 1.5 |
| 2 | Invention | 178706 | 230224 | 10313 | 6 |
| | TRICOR ® | 203088 | 229417 | 16481 | 2 |
| 4 | Invention | 161163 | 218798 | 9740 | 5 |
| | TRICOR ® | 233268 | 276955 | 17075 | 1.51 |
| 5 | Invention | 93427 | 118168 | 4371 | 3.5 |
| | TRICOR ® | 151112 | 161377 | 12941 | 1.5 |
| 6 | Invention | 106373 | 157308 | 4861 | 4 |
| | TRICOR ® | 176647 | 201225 | 13362 | 1.5 |
| 7 | Invention | 36501 | | 946 | 36 |
| | TRICOR ® | 164042 | 184255 | 11427 | 3.5 |
| 8 | Invention | 63421 | 118015 | 2889 | 4 |
| | TRICOR ® | 138052 | 155202 | 12332 | 2 |
| 9 | Invention | 83403 | 95412 | 6613 | 3.5 |
| | TRICOR ® | 137297 | 150641 | 14255 | 2.5 |
| 10 | Invention | 156740 | 200415 | 10077 | 5 |
| | TRICOR ® | 120330 | 214656 | 5181 | 5.05 |
| 11 | Invention | 1494439 | 209197 | 9273 | 9 |
| | TRICOR ® | 237632 | 321628 | 10481 | 5 |
| 12 | Invention | 54000 | 165825 | 1494 | 5 |
| | TRICOR ® | 117399 | 136651 | 8634 | 1.52 |

TABLE 3
Averaged non-transformed pharmacokinetic parameters and
ratios for inventive formulation and TRICOR ®:

| | | | % | 90% Confidence Interval |
|---|---|---|---|---|
| | Inventive | TRICOR ®, | Ratio | (Lower limit, upper limit) |
| Cₘₐₓ | 5822 | 12170 | 0.478 | (0.267, 0.689) |
| (ng/ml) | | | | |
| AUC₀₋ₜ | 105549 | 167270 | 0.631 | (0.494, 0.767) |
| (hr*ng/ml) | | | | |
| AUC_{0-INF} | 158704 | 201640 | 0.787 | (0.665, 0.908) |
| (hr*ng/ml) | | | | |

TABLE 4
Ln-transformed Geometric Means for inventive
dosage and TRICOR ®:

| | Inven- | | % | 90% Confidence Interval |
|---|---|---|---|---|
| | tive | TRICOR ® | Ratio | (Lower limit, upper limit) |
| Cmax | 4540 | 11669 | 0.389 | (0.238, 0.638) |
| (ng/ml) | | | | |
| AUC0-t | 94269 | 162726 | 0.579 | (0.450, 0.746) |
| (hr*ng/ml) | | | | |
| AUC0-INF | 152085 | 194895 | 0.780 | (0.674, 0.903) |
| (hr*ng/ml) | | | | |

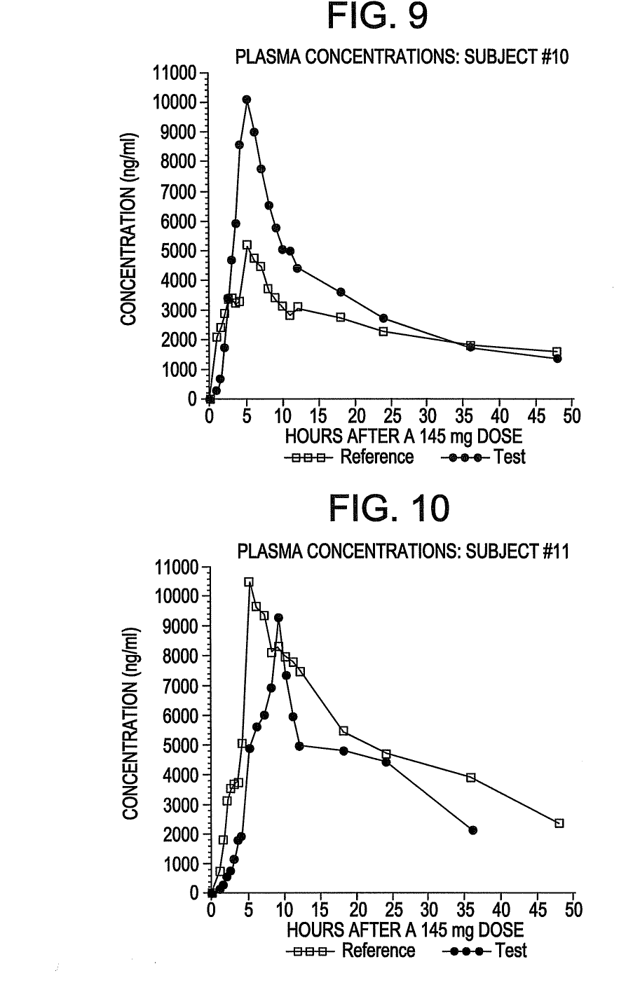

On average, the pharmacokinetic parameters for the inventive dosage form indicate that the tablet tested may not be bioequivalent to TRICOR®. However, the results for several individual subjects suggest that the inventive dosage form can be bioequivalent to TRICOR®. For example, subject 2 (FIG. 2), 9 (FIG. 8), 10 (FIG. 9), and 11 (FIG. 10) exhibit very good absorption compared to TRICOR®.

Without being held to theory, it is believed that the bioavailability of the inventive dosage form may be affected by the tableting process. In order to determine if tableting had an effect on bioavailability, a similar biostudy is being performed on the fenofibrate granules from example 1 in the form of a capsule rather than a tablet. If the inventive fenofibrate capsule has pharmacokinetic parameters that more closely match TRICOR®, then one of several approaches can be used to modify the dosage form of Example 1. In order to facilitate release of the fenofibrate granules from the tablet, additional excipients such as a disintegrant can be added to the tablet. Alternatively, or in addition, prior to tableting, the fenofibrate granules can be coated with a coating composition suitable to protect the fenofibrate granules during the tableting process. Suitable coating compositions for the fenofibrate granules include surfactants, water soluble and water insoluble polymers as described above.

### EXAMPLE 3

### Stability of Fenofibrate Suspension

Fenofibrate suspensions may be formulated as shown in Table 5.

TABLE 5

| Milling suspension compositions | % Weight |
|---|---|
| Fenofibrate | 10 to 22.5 |
| Sodium Phosphate Monobasic Monohydrate | 1 to 5 |
| EUDRAGIT EPO | 1 to 5 |
| Ethanol | 0 to 10 |
| Water | 12 to 57.5 |
| Total | 100 |

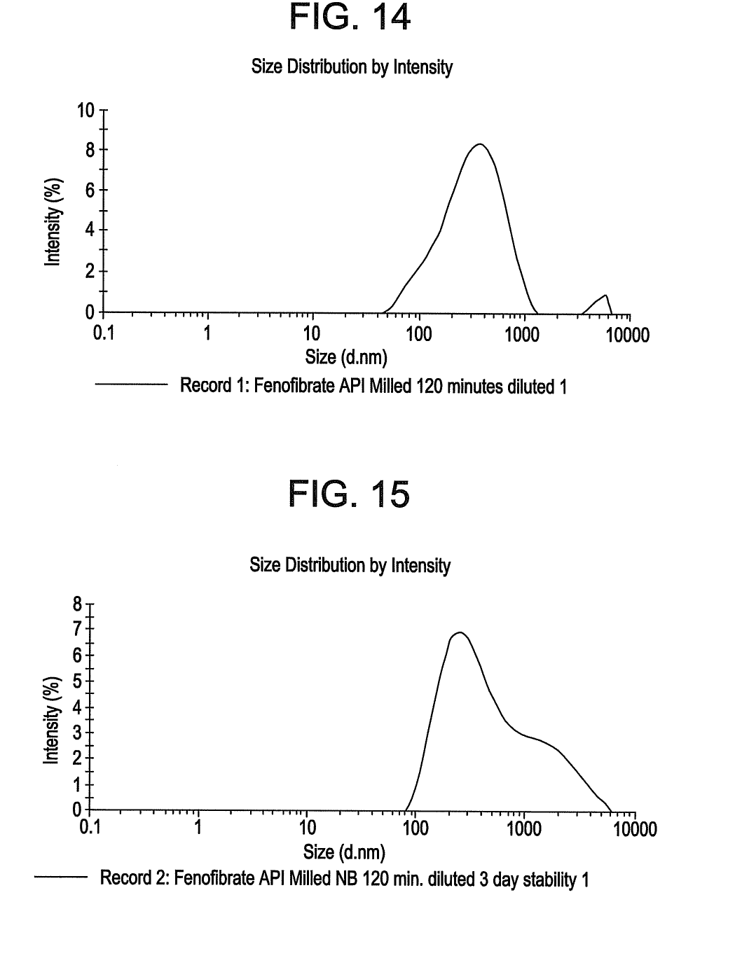

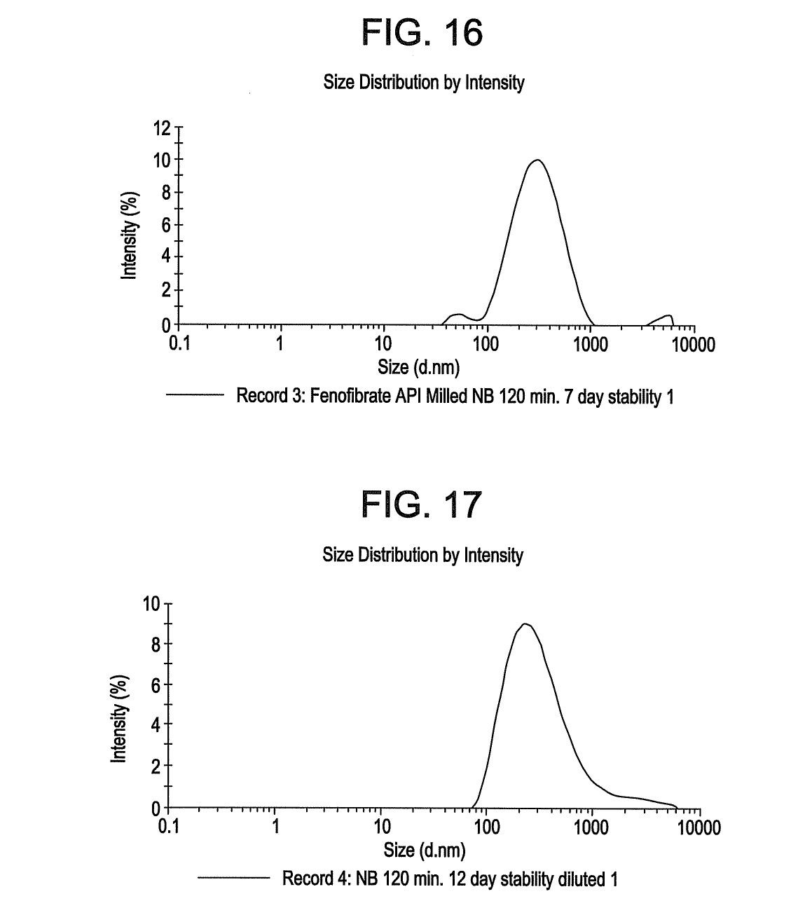

Suspensions falling within the parameters set forth in Table 5 were formulated. The size of fenofibrate nanoparticles in suspension were measured as a function of time. Particle size was measured by Malvern light scattering. FIG. 14 shows the particle size data for an initial time point, shortly after milling. The effective average particle size is about 260 nm. FIG. 15 shows the particle size data for a fenofibrate suspension stored at room temperature for 3 days. The effective average particle size is about 323 nm. FIG. 16 shows the particle size data for a fenofibrate suspension stored at room temperature for 7 days. The effective average particle size is about 254 nm. FIG. 17 shows the particle size data for a fenofibrate suspension stored at room temperature for 12 days. The effective average particle size is about 243 nm. Thus, for 12 days and beyond the fenofibrate particle size in the suspension is stable.

The terms “a” and “an” do not denote a limitation of quantity, but rather denote the presence of at least one of the referenced item. The term “or” means “and/or.” The terms “comprising,” “having,” “including,” and “containing” are to be construed as open-ended terms (i.e., meaning “including, but not limited to”). Unless defined otherwise, technical and scientific terms used herein have the same meaning as is commonly understood by one of skill in the art to which this invention belongs. The endpoints of all ranges directed to the same component or property are inclusive and independently combinable.

Embodiments of this invention are described herein, including the best mode known to the inventors for carrying out the invention. Variations of those preferred embodiments would become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. An active agent composition comprising
active agent nanoparticles having an effective average particle size of less than 2000 nm, and
a particle sequestrant,
wherein the composition comprises no surfactants or phospholipids, and
wherein the active agent has a water solubility of less than 1 mg/ml.

2. The active agent composition of claim 1, wherein the composition is in the form of a suspension.

3. The active agent composition of claim 2, wherein the effective average particle size of the nanoparticles in the suspension changes by no more than 35% within 2 weeks of a first particle size measurement.

4. The active agent composition of claim 1, wherein the active agent nanoparticles and the particle sequestrant are disposed on an inert core particle to form an active agent granulate.

5. The active agent composition of claim 1, wherein the active agent nanoparticles have a D₉₀ of 1500 nm.

6. The active agent composition of claim 1, wherein the active agent nanoparticles have a D₉₀ of 1000 nm.

7. The active agent composition of claim 1, wherein the active agent nanoparticles have a D₉₀ of 500 nm.

8. The active agent composition of claim 1, wherein the active agent comprises sirolimus, thalidomide, or tacrolimus.

9. The active agent composition of claim 1, wherein the particle sequestrant comprises a pH-sensitive copolymer having both hydrophobic (meth)acrylate units and acid-soluble (meth)acrylate units.

10. The active agent composition of claim 1, wherein the particle sequestrant comprises a butyl methacrylate-(2-dimethylaminoethyl) methacrylate-methyl methacrylate copolymer.

11. The active agent composition of claim 4, wherein the inert core particles have an average diameter of 50 to 500 μm.

12. The active agent composition of claim 4, wherein the inert core particle comprises a sugar; microcrystalline cellulose; calcium phosphate, lactose; a polymer; and combinations comprising one or more of the foregoing inert core particles.

13. An active agent suspension comprising
active agent nanoparticles having an effective average particle size of less than 2000 nm, and a particle sequestrant consisting essentially of a butyl methacrylate-(2-dimethylaminoethyl) methacrylate-methyl methacrylate copolymer,
wherein the active agent has a water solubility of less than 1 mg/ml, and
wherein the effective average particle size of the nanoparticles in the suspension changes by no more than 35% within 2 weeks of a first particle size measurement.

14. The active agent suspension of claim 13, wherein the active agent nanoparticles have a D₉₀ of 1500 nm.

15. The active agent suspension of claim 13, wherein the active agent nanoparticles have a D₉₀ of 1000 nm.

16. The active agent suspension of claim 13, wherein the active agent nanoparticles have a D₉₀ of 500 nm.

17. The active agent suspension of claim 13, wherein the active agent comprises sirolimus, thalidomide, or tacrolimus.

18. The active agent suspension of claim 13, wherein the suspension is disposed on an inert core particle to form an active agent granulate.

19. An active agent suspension consisting essentially of
active agent nanoparticles having an effective average particle size of less than 2000 nm, and a butyl methacrylate-(2-dimethylaminoethyl) methacrylate-methyl methacrylate copolymer particle sequestrant,
wherein the active agent has a water solubility of less than 1 mg/ml, and
wherein the effective average particle size of the nanoparticles in the suspension changes by no more than 35% within 2 weeks of a first particle size measurement.
